(19) United States
(12) Patent Application Publication
(10) Pub. No.: US 2024/0285439 A1
(43) Pub. Date: Aug. 29, 2024

(54) LIGHT SHIELD OF A WELDING HELMET AND METHOD OF IMPROVING THE SAME

(71) Applicant: Caleb Michael Moore, Graycourt, SC (US)

(72) Inventor: Caleb Michael Moore, Graycourt, SC (US)

(21) Appl. No.: 18/173,960

(22) Filed: Feb. 24, 2023

(51) Int. Cl. A61F 9/06

(52) U.S. Cl. CPC A61F 9/061; A61F 9/067

(57) ABSTRACT

A method of improving a welding helmet through adding a light shield to selectively cover the light sensor of the welding helmet's window, thereby preventing sensor-based deployment of the automatic window darkening system when the wearer is performing non-welding activity in need of a fully transparent window. The light shield is moveable to an inoperative position so that the automatic window darkening system works as intended when the wearer is welding.

### BACKGROUND OF THE INVENTION

The present invention relates to welding helmets and, more particularly, a method of improving a welding helmet through adding a movable shield to cover the sensor of the welding window, thereby preventing sensor-based deployment of the automatic window darkening system when grinding.

A welding helmet is a type of personal protective equipment used in performing certain types of welding to protect the eyes, face, and neck from flash burn, sparks, infrared and ultraviolet light, and intense heat. Modern welding hoods have automatic window darkening systems for the welding helmet's window, such as an LCD electronic shutter. The automatic window darkening system darkens the welding helmet's window when one or more light sensors detect the bright welding arc.

The advantage of having an automatic window darkening system is that the welder must no longer get ready to weld and then nod their head to lower the helmet over their face. The disadvantage is that while non-welding tasks, where protection of the welding helmet is still needed, such as grinding, the automatic window darkening system darkens the window when the one or more light sensors sense a non-welding light source, such as a grinding spark, even though the welding helmet is flipped out of the way. This hampers the user's vision while performing these non-welding activities.

Current solutions to prevent the unnecessary and detrimental automatic darkening of a welding helmet's window are very heavy and require a lot more parts.

As can be seen, there is a need for a method of improving a welding helmet through a shield adapted to move between an operative position, covering the light sensor(s) of the welding helmet, and an inoperative position so that when welding the automatic window darkening system works as intended.

### SUMMARY OF THE INVENTION

The present invention provides a lightweight, small, low-profile, and durable improvement for welding helmet through providing a shield that pivots between an operative position and an inoperative position, wherein the operative position covers the welding helmet's sensors so no sparks can get to the sensors and trigger the automatic darkening the welding window.

In one embodiment, the present invention is a strip of aluminum that flips down to cover the sensors and allows the user to grind without the lens automatically darkening.

In one aspect of the present invention, a light shield for a welding helmet having one or more light sensors for activating an automatic window darkening system, wherein the light shield is selectively changeable between an inoperative condition and an operative condition that disables the one or more light sensors.

In another aspect of the present invention, the light shield further includes wherein in the operative condition the light shield blocks light from activating the one or more light sensors, wherein the light shield is selectively movable between the operative condition and the inoperative condition by way of pivot points along the welding helmet, wherein the light shield is a strip of opaque material that has a length sufficient so that in the operative condition the light shield extends beyond both sides of the window, wherein the light shield is an approximately six-inch piece of one-inch by one-inch aluminum angle iron, wherein the light shield is U-shaped, wherein the pivot points, respectively, are rivets connected to opposing sidewalls of the welding helmet, wherein the inoperative condition enables the automatic window darkening system.

In yet another aspect of the present invention, the method of improving a welding helmet having an automatic window darkening system activating by one or more light sensors, the method includes connecting a light shield to the welding helmet so that the light shield is selectively movable between an inoperative position and an operative position preventing light from activating the automatic window darkening system, wherein the selective movement is enabled by pivot points attached to the welding helmet.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention, shown in use.

FIG. 2 is a detailed perspective view of an exemplary embodiment of the present invention, shown in use.

FIG. 3 is a detailed perspective view of an exemplary embodiment of the present invention, shown in a stowed condition.

FIG. 4 is an exploded perspective view of an exemplary embodiment of the present invention.

FIG. 5 is a side elevation view of an exemplary embodiment of the present invention, shown in use.

FIG. 6 is a side elevation view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, an embodiment of the present invention provides a method of improving a welding helmet through adding a light shield to cover the light sensor of the welding helmet's window, thereby preventing sensor-based deployment of the automatic window darkening system when the wearer is performing non-welding activity in need of a fully transparent window. The light shield is moveable to an inoperative position so that the automatic window darkening system works as intended when the wearer is welding.

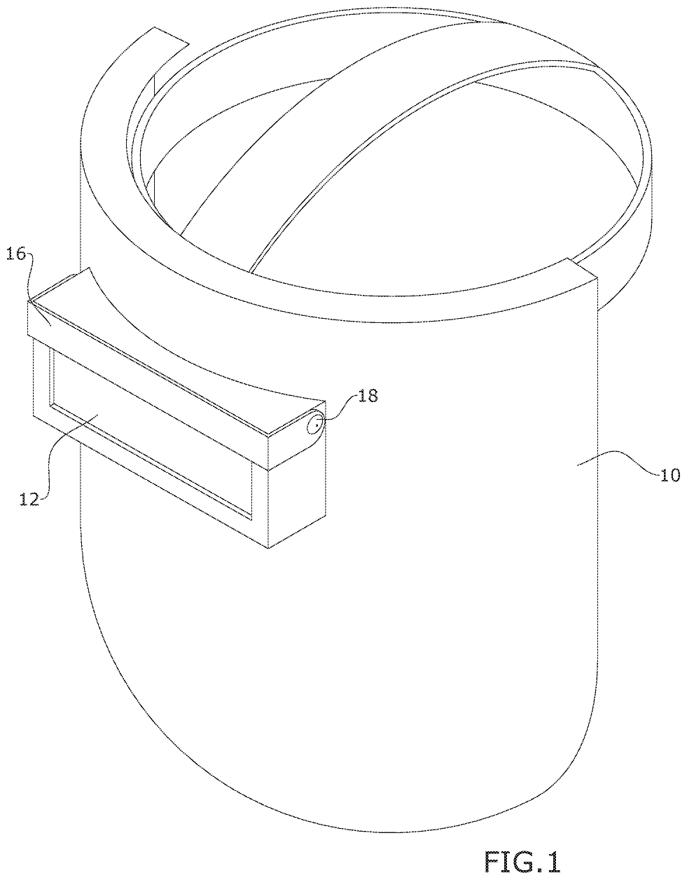

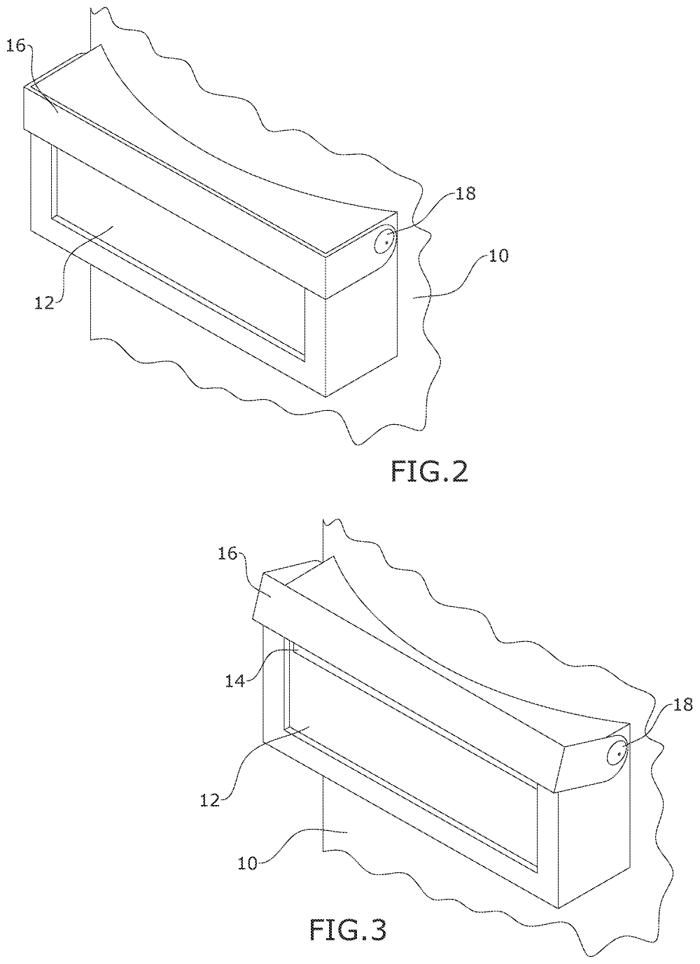

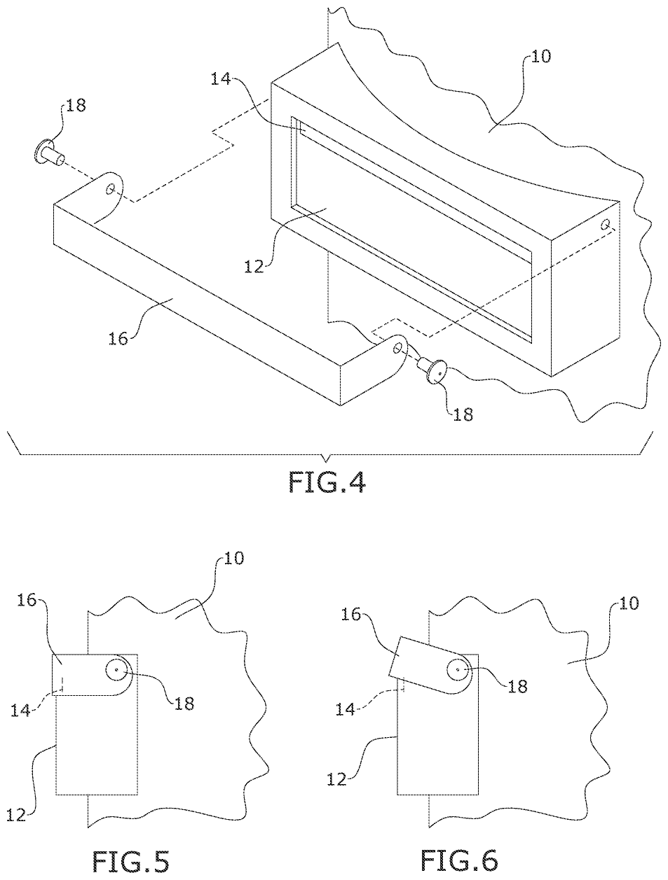

Referring now to FIGS. 1 through 6, the present invention may embody a light shield 16 connected to a welding helmet 10 so that it moves between an inoperative position and an operative position adjacent one or more light sensors 14 so as to prevent activation of the automatic window darkening system of the welding helmet 10.

The one or more light sensors 14 are along or adjacent the helmet's window 12 so that light from arc welding directed to the wearer's field of view will trigger the darkening of that window 12 associated with the automatic window darkening system. Accordingly, so as to prevent such light sensor-based activation, wherever the one or more light sensors 14 are disposed, the operative position of the light shield 16 occupies a space between the one or more light sensors 14 and sources of light (not associated with welding) in the wearer's field of view.

Thus, in some embodiments, the light shield 16 is a strip of opaque material having a length greater than the width of the window 12 so that it projects beyond both (width) sides thereof. In a certain embodiment, the light shield 16 may be an approximately six-inch piece of one-inch by one-inch aluminum angle iron. Though it is contemplated that other material types, shapes, and dimensions are acceptable if they function as disclosed herein. In the inoperative position the light shield does not block light from the one or more light sensors 14, so that they can be activated as originally designed.

The light shield 16 may be connected to the welding helmet 10 by pivot points 18 so that the light shield 16 pivots between the operative and inoperative positions. In one embodiment, the pivot points may be 5/32-inche pop rivets. It is contemplated that other methods of moving the light shield between the operative and inoperative positions are viable. Likewise, the movement between said positions need to me up and down, as the light shield 16 would move from the front to the side in the inoperative position. It is even contemplated that the light shield does not move, but rather can be selectively changed from transparent to opaque, thus effectuating an inoperative condition and an operative condition, respectively.

In certain embodiments, a manufacturer of the light shield 16 may measure, cut, and bend the material to fit the window 12 on the welding helmet 10 so that in the operative position, the light shield 16 occludes light from activating the light sensors 14. A manufacturer may rivet the light shield 15 so that it flips to the inoperative position prior to welding to uncover the sensors 14 and flips down to the operative position when the sensors 14 need to be covered for grinding and other non-welding tasks where the other protective features of the welding helmet 10 are still needed.

As used in this application, the term “about” or “approximately” refers to a range of values within plus or minus 10% of the specified number. And the term “substantially” refers to up to 80% or more of an entirety. Recitation of ranges of values herein are not intended to be limiting, referring instead individually to any and all values falling within the range, unless otherwise indicated, and each separate value within such a range is incorporated into the specification as if it were individually recited herein.

For purposes of this disclosure, the term “aligned” means parallel, substantially parallel, or forming an angle of less than 35.0 degrees. For purposes of this disclosure, the term “transverse” means perpendicular, substantially perpendicular, or forming an angle between 55.0 and 125.0 degrees. Also, for purposes of this disclosure, the term “length” means the longest dimension of an object. Also, for purposes of this disclosure, the term “width” means the dimension of an object from side to side. For the purposes of this disclosure, the term “above” generally means superjacent, substantially superjacent, or higher than another object although not directly overlying the object. Further, for purposes of this disclosure, the term “mechanical communication” generally refers to components being in direct physical contact with each other or being in indirect physical contact with each other where movement of one component affect the position of the other.

The use of any and all examples, or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the disclosed embodiments.

In the following description, it is understood that terms such as “first,” “second,” “top,” “bottom,” “up,” “down,” and the like, are words of convenience and are not to be construed as limiting terms unless specifically stated to the contrary.

It should be understood, of course, that the foregoing relates to exemplary embodiments of the invention and that modifications may be made without departing from the spirit and scope of the invention as set forth in the following claims.

What is claimed is:

1. A light shield for a welding helmet having one or more light sensors for activating an automatic window darkening system, wherein the light shield is selectively changeable between an inoperative condition and an operative condition that disables the one or more light sensors.

2. The light shield of claim 1, wherein in the operative condition the light shield blocks light from activating the one or more light sensors.

3. The light shield of claim 2, wherein the light shield is selectively movable between the operative condition and the inoperative condition by way of pivot points along the welding helmet.

4. The light shield of claim 3, wherein the light shield is a strip of opaque material that has a length sufficient so that in the operative condition the light shield extends beyond both sides of the window.

5. The light shield of claim 4, wherein the light shield is an approximately six-inch piece of one-inch by one-inch aluminum angle iron.

6. The light shield of claim 5, wherein the light shield is U-shaped.

7. The light shield of claim 6, wherein the pivot points, respectively, are rivets connected to opposing sidewalls of the welding helmet.

8. The light shield of claim 7, wherein the inoperative condition enables the automatic window darkening system.

9. A method of improving a welding helmet having an automatic window darkening system activating by one or more light sensors, the method comprising:
connecting a light shield to the welding helmet so that the light shield is selectively movable between an inoperative position and an operative position preventing light from activating the automatic window darkening system.

10. The method of claim 9, wherein the selective movement is enabled by pivot points attached to the welding helmet.
